(19) United States
(12) Patent Application Publication
(10) Pub. No.: US 2008/0146886 A1
(43) Pub. Date: Jun. 19, 2008

(54) SPECULUM

(76) Inventor: David Brian Lucas, Indianapolis, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/958,715

(22) Filed: Dec. 18, 2007

Related U.S. Application Data
(60) Provisional application No. 60/870,508, filed on Dec. 18, 2006.

(51) Int. Cl. A61B 1/303

(52) U.S. Cl. 600/220

(57) ABSTRACT

The opening area of the vagina contains most of its nerve endings; this area is generally much more sensitive to pressure than the inner two-thirds of the vagina. This portion of the vagina is also more constricted than the inner two-third of the vaginal area. When dilated, such as during a vaginal examination, pressure on and near the vaginal opening may increase pressure upon the adjacent urethral opening and the pubic bone, causing increased discomfort without providing additional expansion of the area. The middle area of the vagina is more dilatable, has fewer nerve endings, and more readily deforms when pressure is applied. The disclosed speculum is intended to place little pressure on the somewhat constricted vaginal opening and allow for increased expansion through the middle area, resulting in less discomfort to the patient during examination and maximized viewing area. A speculum and method of use are disclosed. Embodiments of speculum include upper and lower speculum blades. The upper blade may include a relatively wide paddle portion configured to provide a vaginal opening support surface when the speculum is in an open orientation. Another embodiment may include an upper speculum blade with a proximal portion located near the speculum handle which is stepped downward from a distal portion. The proximal portion preferably is configured to expand the vaginal opening less, while distal portion is configured to provide increased dilation or expansion to the vaginal middle area.

This application claims the benefit of U.S. provisional application Ser. No. 60/870,508 filed on Dec. 18, 2006, which is hereby incorporated by reference.

### BACKGROUND

Disclosed embodiments relate to specula. These are described in the context of a gynecological speculum, but are believed to be useful in other applications as well.

Several types of gynecological specula are known. Many earlier types of speculum were designed and manufactured to be sterilizable. More recent types are resin or filled resin types designed and manufactured to be disposable. Some of these later types are made from molded, transparent or semi-transparent plastic to assist in examination. Additionally, some employ optical wave guiding materials either integrally or as attachments, again to assist in examination. There exists a need for an improved speculum with a maximized viewing area and increased comfort during examination.

### BRIEF DESCRIPTION OF THE DRAWINGS

FIG. 1 is a schematic front view of an embodiment of a speculum in an open orientation.

FIG. 2 is a schematic front view of an embodiment of a speculum in a half-open orientation.

FIG. 3 is a schematic front view of an embodiment of a speculum in a closed orientation.

FIG. 4 is an enlarged side view of the upper spreading member shown in FIG. 1.

FIG. 5 is a perspective view of the upper spreading member shown in FIG. 4.

FIG. 6 is a further perspective view of the upper spreading member shown in FIG. 4.

### SUMMARY OF THE DISCLOSURE

The opening area of the vagina contains most of the vaginal nerve endings. As a result, this area is generally much more sensitive to pressure than the inner two-thirds of the vaginal area. This portion of the vagina is also more constricted than the inner two-third of the vaginal area. When dilated, such as during a vaginal examination, pressure on and near the vaginal opening may increase pressure upon the adjacent urethral opening and the pubic bone, causing increased discomfort without providing additional dilation. The middle area of the vagina is more dilatable, has fewer nerve endings, and can more readily deform when pressure is applied. The disclosed speculum may minimize pressure on the somewhat constricted vaginal opening and allow for increased dilation through the middle area. This may result in less discomfort to the patient during examination, while maximizing the viewing area of the cervix and vaginal wall.

A speculum is disclosed. Embodiments of speculum include upper and lower speculum blades. The upper blade may include a relatively wide paddle portion configured to provide a vaginal opening support surface when the speculum is in an open orientation. Another embodiment includes an upper speculum blade with a proximal portion located near the speculum handle which is stepped downward from a distal portion. The proximal portion may be configured to expand less, providing more comfort, to the vaginal opening, while the distal portion may be configured to provide increased expansion to the vaginal middle area.

A method for using the speculum is also disclosed.

### DETAILED DESCRIPTION OF THE ILLUSTRATED EMBODIMENTS

For the purposes of promoting an understanding of the principles of the disclosure, reference will now be made to the embodiment illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

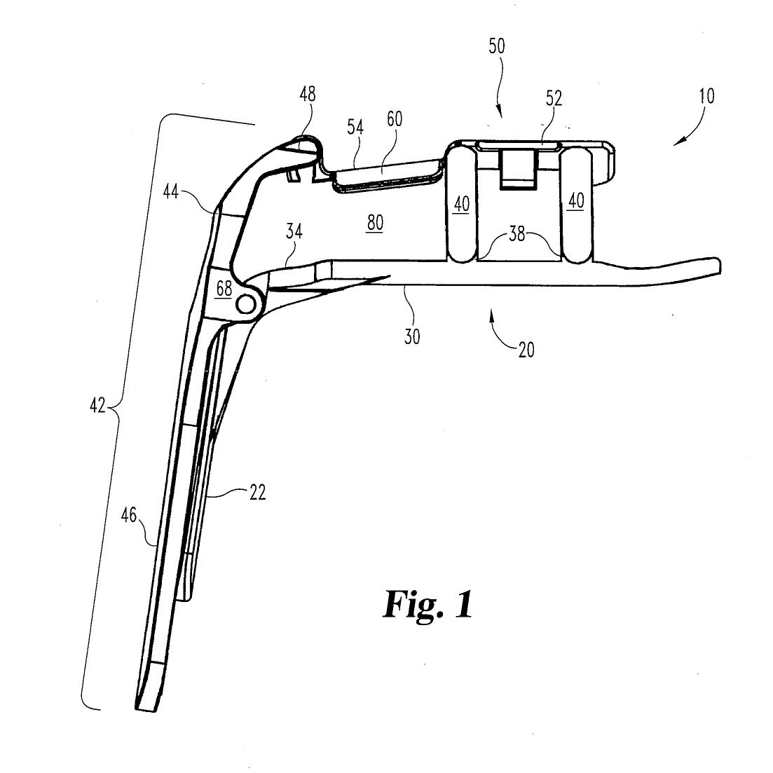

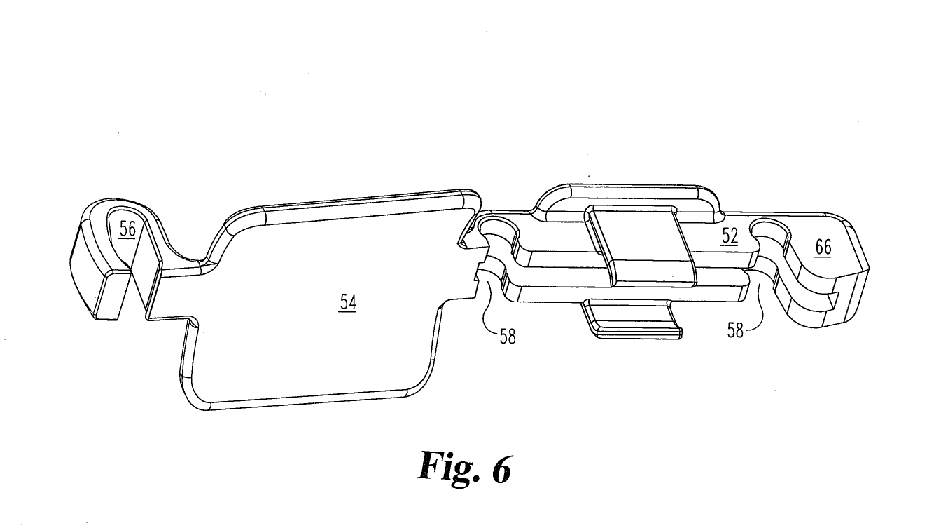

As shown in FIGS. 1-6, the present disclosure relates to a speculum 10. While not intending to be bound by theory or limited to a speculum for use in vaginal examination, background on the anatomy and structure of the vagina may be helpful in understanding certain aspects of the present disclosure. Although the vagina is generally thought of as a canal, the vagina is more accurately regarded as “potential” space. The walls of the vagina are normally in contact with each other, e.g. collapsed. When something enters the vagina, the body must make room for it. The vagina has three parts: the opening area, the middle area and the uterine extremity.

The opening area of the vagina (which is used herein to refer to the actual opening as well as the area near the opening) contains most of the vaginal nerve endings. As a result, this area is generally much more sensitive to pressure than the inner two-thirds of the vagina. This portion of the vagina is also rather constricted. When expanded or dilated, pressure on and near the vaginal opening increases pressure upon the urethral opening and the pubic bone, as these structures are adjacent to the opening area. In a non-pregnant patient, the pubic bone deforms very little, and therefore, transmission of increased pressure to the bone will result in increased discomfort to the patient, with little gain in vaginal dilation in the opening area.

The middle area of the vagina can be easily and further dilated and has fewer nerve endings than the opening area. It is also adjacent to structures, such as the bladder, which can more readily deform when pressure is applied. The vagina typically narrows near its uterine extremity. The design of speculum 10 is intended to account for these structural characteristics. In use, speculum 10 preferably places little pressure on the somewhat constricted vaginal opening, allowing for increased dilation through the middle area and approaches the uterine extremity only with its farthest reaching point, tongue 36. This results in less discomfort to the patient during examination, while maximizing the viewing area of the cervix and vaginal wall.

Referring generally to FIGS. 1-6, there is shown an embodiment of a speculum 10. As illustrated, speculum 10 has a lower spreading member 20, which includes a handle 22 and a speculum blade 30. Blade 30 may be further divided into a rail portion 32, a connector 34 and a tongue 36. In the illustrated embodiment, member 20 is connected via ribs 40 to an upper spreading member 50. Member 50 is further connected to handle 42. Handle 42 may further include a C-shaped upper portion 44 and a gripping lower portion 46. Member 50, as illustrated, is configured to include a paddle portion 54 and a rail portion 52. This disclosure, and specifically the described embodiments of speculum 10, represents a variation on the embodiment disclosed in U.S. Pat. No. 6,740,031, which is herein incorporated by reference in its entirety.

In the embodiment as shown in FIGS. 1-3, a lower spreading member 20, composed of a handle 22 and a blade 30, is a single, continuous piece. A rail portion 32 forms the majority of the blade 30, with the tongue 36 at an end distal to the handle 22 and the connector 34 at an end proximal to the handle 22. As shown, the rail portion 32 is generally long and thin and has a substantially rectangular cross-section.

In the illustrated embodiment, rail portion 32 has retaining area 38 enabling rail portion 32, and thus spreading member 20, to connect, via ribs 40, to upper spreading member 50. Retaining area 38 may be shaped or configured to allow ribs 40 to be retained through a snap-fit ring retaining mechanism. Retaining area 38 might also be otherwise configured, so long as ribs 40 make contact and are retained.

As illustrated, rail portion 32 extends at one end to tongue 36, which, in the option shown, is semi-circular and generally convex. Rail portion 32 is connected at its other end to connector 34. Connector 34 may define an approximately 90° turn or a greater or lower angle as desired, connecting blade 30 to handle 22. Connector 34 may be sized and configured to allow for a stable connection to rail portion 32 and handle 22, as well as providing a tailored fit to any meatus or incision in which speculum 10 will be placed. As shown, handle 22 is long and thin and runs substantially perpendicular to rail portion 32. Handle 22 may contain a gripping surface (not shown), such as a roughened or raised area. Handle 22 is depicted as being shorter than handle 52, though handle 22 could be of an equal length or longer if so desired.

While lower spreading member 20 has been illustrated and described, it should be understood that variations in member 20 are contemplated within this disclosure. For example, rail portion 32 could have a different cross-section—such as square, oval or round, or it could have a rounded lower surface. Tongue 32 might be longer, shorter, flat, less convex or more convex than depicted. Tongue 32 might also have a more squared or oval shape, be otherwise regularly shaped (such as having symmetrical undulations or curves) or have an irregular shape.

Link members, depicted as ribs 40, are generally shown as being circular portions, defining a circular cross-section. In the illustrated embodiment, two ribs 40 are shown, which are parallel and connect to rail portion 32 of member 20 and rail portion 58 of member 50. The ribs are generally half-circles which latch onto either side of rail portion 32 and fit through a portion of rail portion 58. In the illustrated embodiment, ribs 40 are pivotally mounted on rail portion 32 and are received in a hole (not shown) on each side of rail portion 32. In the illustrated embodiment, ribs 40 are mounted through a snap-fit arrangement in a hole on each side of rail portion 32 and ribs 40 are received through a semicircular space in rail portion 58. This arrangement provides secure attachment for ribs, while allowing them to pivotally move from a flat position (when speculum 10 is in a closed orientation) to an upright position (when speculum 10 is in an open position) and all positions in between. Other means for securing ribs to rail portion 32 and rail portion 58 while allowing for pivotal movement are contemplated. For example, ribs 40 could be secured to rail portions 32 and 58 through a hole and pin system, magnetic means or through a friction fit.

Other designs for ribs 40 also are contemplated. There could alternatively be only one rib 40 or more than two ribs 40. Ribs 40 could also be other than circular. They could also have a non-circular cross-section. For example, ribs 40 could have a generally square or rectangular configuration with a generally circular or otherwise shaped cross-section. They could also be made of a more deformable material than the rest of speculum 10 or they could be made with portions being more deformable. Ribs 40, as described more fully below, are generally configured to lie somewhat flat when speculum 10 is in a closed orientation and to provide support and help maintain an open orientation when the speculum is open.

In addition to enabling speculum 10 to move from closed to open orientations, ribs 40 may also provide lateral vaginal wall retention when speculum 10 is in an open orientation. This support is especially important in examination of an overweight patient or a patient who has given birth multiple times, as connective and other tissue may be more pronounced in these patients. If lateral support of the vaginal walls is not provided, this tissue could intrude and might obscure both the view of the cervix (located at the uterine extremity of the vagina) and views of the vaginal walls. Visual examination of these areas may be important in correctly diagnosing diseases, such as cancer, in their earliest stages. In addition to the circular structure of ribs 40, which provide lateral vaginal wall support, the location of the ribs may also confer benefits. For example, rib 40 being placed near paddle portion 54 may enable rib 40 to support the vaginal walls at the point where the vagina transitions from its opening area to its middle area. This positioning is intended to provide critical support for the vaginal wall at one of the first places where increased dilation is possible and desired: the beginning of the middle area.

An upper spreading member 50 is connected to the handle 42. As illustrated, the handle 42 includes a C-shaped upper portion 44 and a lower portion 46. The upper portion 44 connects the lower spreading member 20 and the upper spreading member 50 and may further include a upper arm 48 and a lower arm 68. The upper arm 48 may include a bar (not shown) or other connection means for pivotally attaching to the upper spreading member 50. The lower arm 68 may include a hole 70 or other means for pivotally attaching to the connector 34 of lower spreading member 20. As shown, the lower arm 68 may be pivotally mounted to the connector 34 of the lower spreading member 20 by one or more pivot pins inserted through the arm 68, the connector 34 and back through the arm 68. As illustrated, a lower portion 46 of the handle 42 is long and thin and generally shaped to allow it to be easily gripped. The lower portion 46 could be further equipped with a gripping area (not shown) having a roughened, raised or otherwise modified area to enhance gripping. Upper portion 44 defines an opening, which is rearward facing, allowing access to channel 80 when the speculum is open.

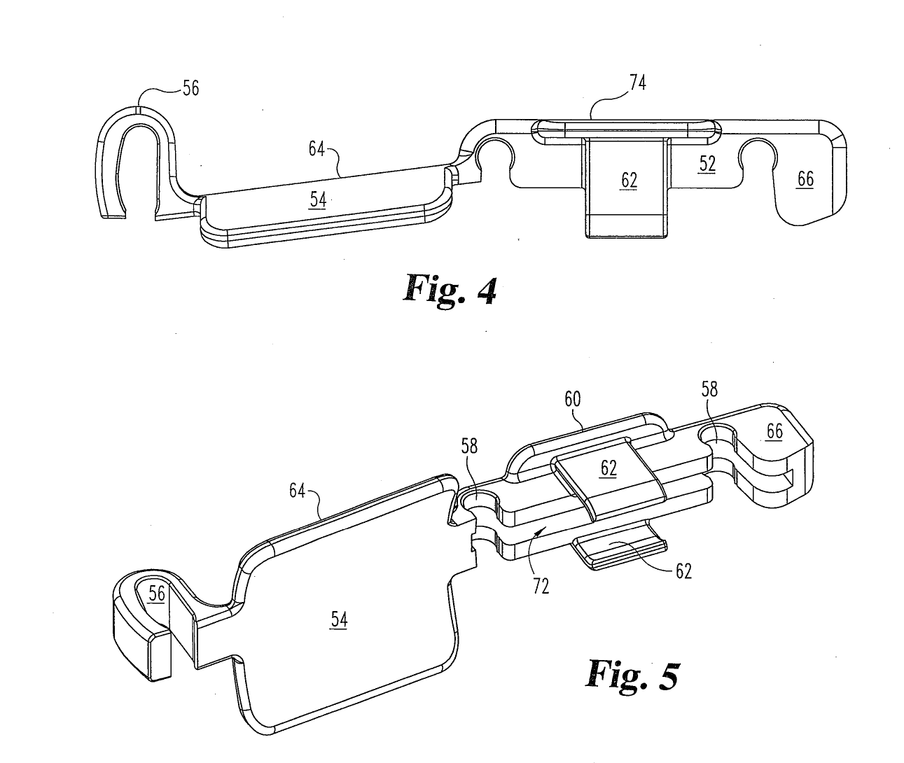

As illustrated in FIGS. 4-6, upper spreading member 50 has a horseshoe-shaped attaching portion 56, a proximal paddle portion 54, and a distal rail portion 52. Rail portion 52 further includes openings or retaining areas 58, which may connect to ribs 40, a widened area, or a second paddle portion 60, clamp 62 and lip 66. In general, rail portion 52 has a similar shape to rail portion 32, being generally long and thin shaped. As illustrated in FIG. 5, rail portion 58 may have a channel 72 and a top face 74. Attaching portion 56 and retaining areas 58 are generally parallel to one another. Attaching portion 56 defines an elongate opening to connect to upper arm 48, which allows the pivot point to translate slightly during movement.

Paddle portion 54 is generally flat, having a top face 64, a bottom and two sides. The sides of the paddle portion are generally thin and uniformly sized. Bottom is generally correspondingly shaped to top face 64. As seen in FIG. 5, paddle portion 54 may be somewhat curved and have a cupped or concave (with respect to channel 80) form. Illustrated paddle portion 54 is generally rectangularly-shaped with rounded edges and corners. Paddle portion 54 has a top face 64. The general shape of paddle portion 54 may provide increased comfort during vaginal examination. The width of the paddle portion's top face 64 combined with generally thinness of the sides of the paddle portion 54 allows pressure to be distributed across its entire surface area. Its rounded corners and generally cupped shape also enable it to accommodate the shape of the opening area of the vagina. By distributing pressure exerted along an increased surface area, mirroring the shape of the area into which it is to be placed and eliminating potential sharp areas, such as corners, paddle portion 54 is sized and shaped to accommodate the vaginal opening, a more constricted and less deformable area than other portions of the vagina, such as the middle area.

As seen in FIG. 4, paddle portion face 64 is stepped downward in height or lower relative to the upper face 74 of rail portion 52 and attaching portion 56. This may provide an additional benefit during examination. Paddle portion face 64 may more properly conform to the depth of the opening area of the vagina. For example, this stepped down portion of speculum 10 may conform to the reduced diameter found in the opening area of the vagina. Unlike traditional speculum models, which generally have a more uniform inter-blade distance, the stepped downward area of paddle portion face 64 accomodates the opening area of the vagina while the upper face 74 of rail portion 52, along with second paddle portion 60, with its stepped up configuration can more readily accommodate the middle area of the vagina, where increased dilation is possible with minimal discomfort.

Paddle portion 54 has been described as depicted in the embodiment, but it should be understood that paddle portion 54 is not limited to this configuration. Paddle portion 54 could be generally circular, oval, square or irregularly configured. Additionally, paddle portion 54 could be more or less cupped or concave, or even flat. It is also contemplated that paddle portion 54 could be shaped more similarly to rail portion 32—generally longer, and thinner than depicted, or it could be a series of rail portion-like portions spanned with a second material, such as webbing. Additionally, it could be made of material with a uniform thickness or a variable thickness.

Second paddle portion 60 has a generally flatter appearance, and is wider than rail portion 58. As illustrated in FIG. 5, second paddle portion 60 is generally not as wide or large as paddle portion 54. It is contemplated that in certain embodiments, second paddle portion 60 might be sized and configured similarly to paddle portion 54. As with paddle portion 54, second paddle portion 60 has two sides that are generally uniformly sized and relatively thin compared to the face of paddle portion 60. The top of second paddle portion 60 may be somewhat curved or cupped. As depicted in FIG. 5, second paddle portion 60 is generally rectangularly-shaped with rounded edges. Paddle portion 60 has been described as depicted in the embodiment, but it should be understood that paddle portion 60 is not limited this configuration. It could be generally circular, oval, square or irregularly configured. Additionally, paddle portion 60 could be more or less cupped or concave, or even flat. It is also contemplated that paddle portion 60 could be shaped more similarly to rail portion 32—generally longer, and thinner than depicted, or it could be a series of rail portion-like portions spanned with a second material, such as webbing. Additionally, it could be made of material with a uniform thickness or a variable thickness.

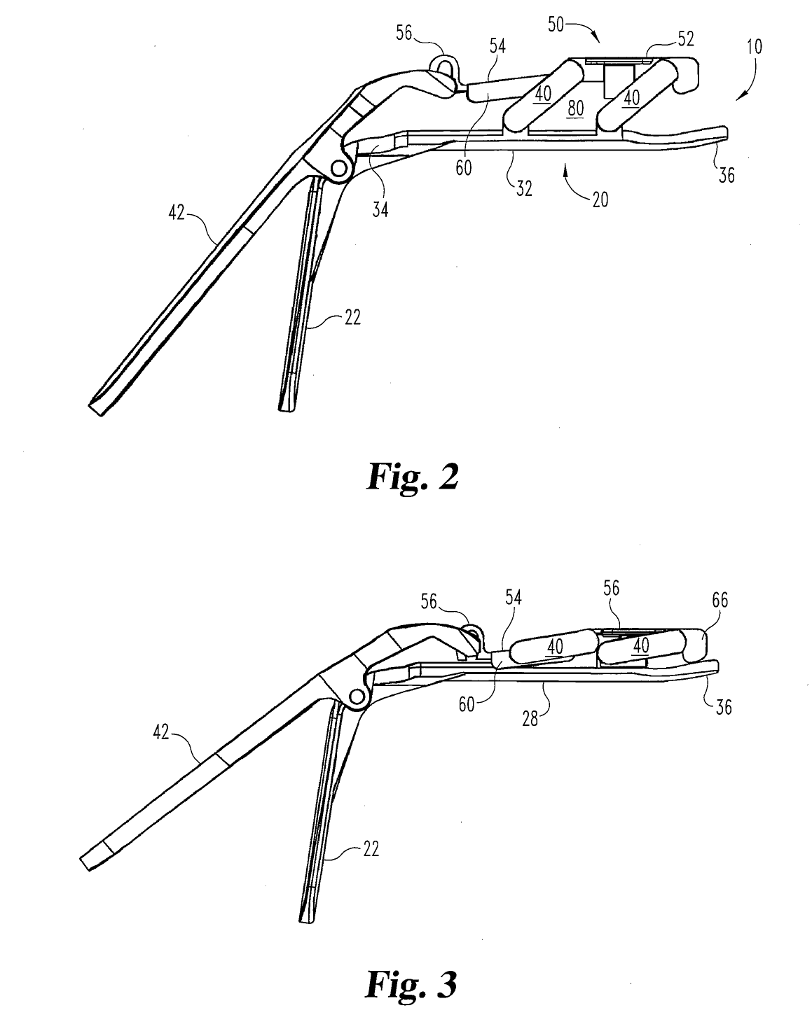

In use, speculum 10 can be moved from a closed orientation (FIG. 3) to a half-open orientation (FIG. 2) to an open orientation (FIG. 1). In the closed orientation, member 20 and member 50 are generally adjacent one another, with ribs 40 “folded” in a generally flat position, sandwiched between blade 30 and member 50. Lip 66 of member 50 touches tongue 36 of member 20 to limit the movement. Attaching portion 56 of member 50 moves adjacent to rail portion 32. Likewise, upper arm 48 of handle 42 is nearly touching rail portion 32. This closed, compressed orientation allows blade 30 and member 50 of speculum 10 to have a thin profile.

In order to move speculum 10 from its closed orientation to its half open orientation, pressure is applied to handle 42, and it is moved downwardly and inwardly towards handle 22. Handle 22 and handle 42 may be gripped and an adequate amount of pressure applied. Varying the amount of pressure allows a user to determine the rate at which speculum 10 moves from its closed orientation through its half open orientation and to its open orientation. Speculum 10 may be moved only to half open and then back to closed or through any other permutation of closed, half open and open orientations desired.

In its half open orientation, lower spreading member 20 and upper spreading member 50 are no longer touching or almost touching one another. Ribs 40 extend to a roughly 45° angle. Lip 66 of member 50 ceases to touch tongue 36 of member 20 and lip 66 is more proximate to handle 22 than in the closed orientation. Attaching portion 56 has moved up and back with respect to its closed orientation. Upper arm 48 of handle 42 is engaged with attaching portion 56 in a pivotal motion, thereby enabling attaching portion 56, and all of member 50, to exhibit translational, as well as vertical movement. In the half open orientation, passageway 80 is created—the passageway being defined by the space created between member 20 and member 50 and the hollow interior of ribs 40.

In its fully open orientation, lower spreading member 20 and upper spreading member 50 have a maximum space between them and passageway 80 is substantially at its greatest diameter. Ribs 40 are extended to a roughly 90° angle. Optionally, ribs 40 may be extended to slightly beyond a 90° angle to lock the speculum open. Lip 66 of member 50 has moved translationally and vertically further away from tongue 36 of member 20. Attaching portion 56 has moved further up and back than with respect to its closed orientation. Upper arm 48 of handle 42 continues to be engaged with attaching portion 56 in a pivotal motion, thereby enabling attaching portion 56, and all of upper spreading member 50, to exhibit translational and vertical movement. At the fully open or slightly beyond orientation, attaching portion 56 of upper spreading member 50 and upper arm 48 of handle 42 may be in an over-center position with respect to lower spreading member 20 thereby locking portions 20 and 58 (as well as 50) in this position. This can be best appreciated with reference to FIG. 1.

The use of the speculum 10 will now be described in the context of a vaginal examination. This use should not be considered to limit the usage of speculum 10, but rather provides a description of a specific context for its usage. A patient will generally lie on her back on an examination table with her feet raised and supported by stirrups. A lamp is often turned on to allow adequate lighting for the examination. The speculum 10 may be warmed (with water or otherwise) and/or lubricated with a lubricant prior to insertion. The speculum 10 is then gently inserted into a vaginal cavity by first positioning the speculum 10, in its closed orientation, in the vaginal opening. The thin profile of the speculum 10 in its closed orientation may allow this to insertion to occur with little to no vaginal pressure, either in the vaginal opening or in the vaginal cavity.

Once correctly positioned inside the vaginal cavity, selective pressure is applied to handle 42 and handle 22 such that speculum 10 moves from its closed orientation through its half open orientation and eventually into its open orientation. Top edge 64 of paddle portion 54 of upper spreading member 50 will slowly come into contact with the opening of the vaginal wall as speculum 10 is moved from its closed to open orientation. The rounded corners, increased surface area and concavity of paddle portion 54 allow it to more comfortably contact this area, thereby conforming appropriately to the less dilated, less pressure absorbing portion of the vagina. The pressure of the contact of paddle portion 54 with the vaginal wall is distributed throughout its increased surface area, and therefore pressure in any specific area, such as on a nerve ending, is minimized.

Further, with the top face 64 of the paddle portion 54 being lower or closer to the lower spreading member 20 than top face 74 of rail portion 52, there is decreased “spreading” required at the vaginal opening, and thus decreased discomfort when, for top face 64 of paddle portion 54 to be fully engaged in the vaginal wall. In this orientation, the viewing area provided by passageway 80 is maximized due to the position of top face 74 of rail portion 52, and ribs 40.

The blades of speculum 10 may aid in expanding the viewing area and retaining the vaginal walls. Ribs 40 of speculum 10 may further retain vaginal sidewalls, providing increased lateral support to vaginal sidewalls. As paddle portion 54 is generally positioned in the opening area of the vagina, and paddle portion 60 is generally positioned in the middle area of the vagina, if rib 40 is placed adjacent paddle portion 54, upon insertion and opening of speculum 10, rib 40 will be appropriately placed in an area where the vagina can be more dilated (the middle area) and the rib will provide necessary dilation and support at the earliest stage that the vaginal structure will comfortably accommodate it.

Increased support of the vaginal walls allows for maximized viewing area of the cervix and vaginal walls. This maximized viewing area may be achieved while the speculum 10 contacts as little vaginal surface area as necessary. While specula can be made of transparent materials, those materials still have an inherent percentage of haze associated with them. Additionally, the vaginal tissue may be most accurately viewed in its “natural” state, rather than through an instrument. As such, a speculum which prevents vaginal wall collapse, thereby maximizing viewing area, while occupying as little surface area as necessary to do so may be preferable.

Once in its fully open orientation, attaching portion 56 of upper spreading member 50 and upper arm 48 of handle 42 can be in an over-center position with respect to lower spreading member 20. If this is the case, portions 20 and 58 (as well as 50) are locked in this position, holding speculum 10 in an open orientation. Once locked in an open orientation, speculum 10 will remain in place until handles 22 and 42 are separated. In the locked position, a doctor or medical provider can release handles 22 and 42 and speculum 10 will remain in place, allowing the medical provider “hands free” performance.

By looking through passageway 80, the vaginal walls and cervix may be checked for damage, growths, inflammation, unusual discharge, or discoloration. A small brush or spatula may be inserted through passageway 80, allowing the collection of cervical cells (“a Pap smear”). Additionally, a sample of the cervical mucus may be taken with a swab for further testing, such as to determine the presence of sexually transmitted diseases. Additional examination or treatment procedures may also be performed. When examination and/or treatment are complete, speculum 10 can be collapsed into its closed orientation by squeezing and allowing handles 42 and 22 to release to their closed orientation. Speculum 10 may then be removed from the vaginal cavity.

Embodiments of the speculum may be constructed from any suitable material or combinations of materials such as filled and/or unfilled resin materials, illustratively to permit its components to be conveniently formed by, for example, injection molding and assembled by, for example, snapping together. Additionally, materials may be transparent or semi-transparent, thereby visually aiding in examinations conducted using the speculum. Optical waveguiding may also be incorporated in the design and construction of this speculum, further aiding in examination.

While the illustrated embodiments have been detailed in the drawings and foregoing description, the same is to be considered as illustrative and not restrictive in character, it being understood that only the preferred embodiment has been shown and described and that all changes and modifications that come within the spirit of the invention are desired to be protected. The articles “a”, “an”, “said” and “the” are not limited to a singular element, and include one or more such elements.

What is claimed is:

1. A speculum, comprising:
a) a lower speculum blade, wherein said lower speculum blade has a first handle portion;
b) an upper speculum blade;
c) a second handle portion movably mounted to said lower speculum blade and said upper speculum blade, wherein movement of said second handle portion towards said first handle portion actuates movement of said speculum from said compressed orientation to said open orientation
d) at least one link member mounted between said lower speculum blade and said upper speculum blade to allow said speculum to move from a compressed orientation to an open orientation;
said upper speculum blade further including a rail portion and a paddle portion located between said second handle portion and said rail portion, said paddle portion having a top, a bottom and a first side and a second side, said top of said paddle portion being wider than said rail portion and being configured to provide a vaginal opening support surface when speculum is in an open orientation.

2. The speculum of claim 1 wherein said top of said paddle portion is substantially rectangular.

3. The speculum of claim 2 wherein said paddle portion is concave downward.

4. The speculum of claim 3 wherein said paddle portion has rounded corners.

5. The speculum of claim 4 wherein a link member connects to said upper speculum blade between said paddle portion and said rail portion, thereby enabling the link member to provide increased lateral vaginal wall retention when speculum is in an open orientation.

6. The speculum of claim 5 wherein said top of said paddle portion is similarly shaped to said bottom of said paddle portion.

7. The speculum of claim 6 wherein said first side and said second side are identical.

8. A method of using a speculum comprising the steps of:
a) providing a speculum having a lower speculum blade and an upper speculum blade wherein said upper speculum blade includes a paddle portion, said paddle portion having a top surface to provide a vaginal opening support surface when said speculum is in an open orientation;
b) inserting said first and second speculum blades into a vaginal examination site; and
c) opening said upper and lower speculum blades inside a vaginal examination site to dilate the examination site wherein said paddle portion is placed to provide a vaginal opening support surface dispersing force over an area spread across the width of the opening.

9. The method of claim 8 wherein said paddle portion of said upper speculum blade is substantially rectangular.

10. The method of claim 9 wherein said paddle portion is concave downward.

11. The method of claim 10 wherein said paddle portion has rounded corners.

12. The method of claim 11 wherein said upper speculum blade further includes a rail portion wherein said rail portion enters the examination site prior to said paddle portion and wherein said paddle portion is stepped downwards in height as compared to said rail portion and said rail portion is configured to provide maximal dilation of a middle area of a vaginal examination area while said paddle portion is configured to support a vaginal opening and provide as little pressure as necessary on said vaginal opening area.

13. The method of claim 12 wherein said lower speculum blade further includes a tongue, said tongue being the first portion of said speculum to enter a vaginal examination site wherein said tongue is configured to provide maximal visualization of a uterine extremity area of a vaginal examination site by retracting obscuring tissue adjacent the cervix.

14. The method of claim 13 wherein said tongue is wider than the rest of said lower speculum blade.

15. The method of claim 14 wherein said tongue is semi-circular and generally convex upwards.

16. The method of claim 15 wherein said speculum further comprises at least one link member mounted between said lower speculum blade and said upper speculum blade to support said upper and lower speculum blades in an spatially pre-determinated open configuration.

17. The method of claim 16 wherein said at least one link member is located on said upper speculum blade between said paddle portion and said rail portion, and when said speculum blades are in an open configuration said at least one link member is positioned in a vaginal examination site to provide lateral vaginal wall support at a transition area between said vaginal opening area and said vaginal middle area.

18. A speculum, comprising:
a) a lower speculum blade, wherein said lower speculum blade has a first handle portion;
b) an upper speculum blade;
c) a second handle portion movably mounted to said lower speculum blade and said upper speculum blade, wherein movement of said second handle portion towards said first handle portion actuates movement of said speculum from said compressed orientation to said open orientation
d) at least one link member mounted between said lower speculum blade and said upper speculum blade to allow said speculum to move from a compressed orientation to an open orientation;
said upper speculum blade further including a proximal portion located near said second handle portion, and a distal portion, said proximal portion being stepped downward from said distal portion wherein said proximal portion is configured to dilate a vaginal opening and said distal portion is configured to dilate and support a vaginal middle area.

19. The speculum of claim 18 wherein said proximal portion and said distal portion are approximately parallel to each other.

20. The speculum of claim 19 wherein said proximal portion is a paddle portion and said distal portion is a rail portion rail, said paddle portion located between said second handle portion and said rail portion, said paddle portion having a top, a bottom and a first side and a second side, said top of said paddle portion being wider than said rail portion and being configured to provide a vaginal opening support surface when speculum is in an open orientation.
